SEQUENCE LISTING (1 sequence)

SEQ ID NO: 1 - protein, 5 aa, synthetic construct
VPGKG

(19) United States
(12) Patent Application Publication
(10) Pub. No.: US 2024/0398892 A1
(43) Pub. Date: Dec. 5, 2024

(54) HEPARIN NEUTRALIZING PEPTIDE, HEPARIN NEUTRALIZER, AND USE THEREOF

(71) Applicant: DWI - Leibniz-Institut für Interaktive Materialien e. V., Aachen (DE)

(72) Inventors: Andreas HERRMANN, Aachen (DE); Lifei ZHENG, Wenzhou,Zhejiang (CN); Han DING, Wenzhou,Zhejiang (CN); Qing LIU, Wenzhou,Zhejiang (CN)

(73) Assignee: DWI - Leibniz-Institut für Interaktive Materialien e.V., Aachen (DE)

(21) Appl. No.: 18/700,471

(22) PCT Filed: Oct. 13, 2022
(86) PCT No.: PCT/EP2022/078547

§ 371 Date: Apr. 11, 2024

(30) Foreign Application Priority Data
Oct. 14, 2021 (CN) 2021111959642

(51) Int. Cl. A61K 38/16; C07K 14/00

(52) U.S. Cl. CPC A61K 38/16; C07K 14/00

(57) ABSTRACT

The present disclosure provides a heparin neutralizing peptide, a heparin neutralizer, and use thereof, and belongs to the technical field of biomedicine. The heparin neutralizing peptide includes tandem repeating units. The repeating units each have an amino acid sequence shown in SEQ ID NO: 1, specifically VPGKG, where lysine (K) has a positive charge, the repeating units each have a positive charge, and the heparin neutralizing peptide has a plurality of positive charges. The heparin neutralizing peptide with positive charges can bind to a heparin with negative charges through electrostatic interaction to form macromolecular substances, and the macromolecular substances are eliminated from the body by liver metabolism, so that the anticoagulation of the heparin is shielded. Furthermore, the heparin neutralizing peptide provided in the present disclosure can significantly reduce toxic and side effects of neutralizing heparin and reduce hemolysis compared with protamine sulfate in the prior art.

### TECHNICAL FIELD

The present disclosure relates to the technical field of biomedicine, in particular to a heparin neutralizing peptide, a heparin neutralizer, and use thereof.

### BACKGROUND

Heparin is a highly sulfonated natural glycosaminoglycan that is widely used as an anticoagulant material. The anticoagulant ability of the heparin comes from the ultrastrong binding effect of antithrombin III. After antithrombin III binds to heparin, it undergoes a configurational change and is thus activated, which can increase the ability to bind to thrombin and anti-factor Xa by 1,000 times and significantly reduce the coagulation response. Therefore, heparin is widely used to treat and prevent pulmonary embolism, deep vein thrombosis, and myocardial infarction. Also, heparin is widely used in cardiovascular surgery, postoperative care, blood collection tubes and hemodialysis machines.

The coagulation response in vivo needs to be restored after cardiovascular surgery or blood transfusion, so the residual heparin in vivo must be promptly and completely neutralized. Meanwhile, excessive heparin can produce toxic and side effects, for example, bleeding and thrombocytopenia. Currently, protamine sulfate is the only approved medicinal heparin neutralizer. However, protamine sulfate has certain toxic and side effects, for example, causing hemolysis, hypotension, slow heart rate, and very serious allergic reactions. The complex formed by binding of protamine sulfate to heparin has been further shown to have systemic toxicity.

### SUMMARY

An objective of the present disclosure is to provide a heparin neutralizing peptide, a heparin neutralizer, and use thereof. The heparin neutralizing peptide provided in the present disclosure has weaker toxic and side effects than a common heparin neutralizer, protamine sulfate.

To achieve the above objective, the present disclosure provides the following technical solutions:

The present disclosure provides a heparin neutralizing peptide, including tandem repeating units, where the repeating units each have an amino acid sequence shown in SEQ ID NO: 1.

Preferably, the number of the repeating units may be 18, 36, 72 or 144.

The present disclosure provides a heparin neutralizer, including a buffer and the heparin neutralizing peptide according to the foregoing solution.

Preferably, the buffer includes a phosphate-buffered saline (PBS).

Preferably, a concentration of the heparin neutralizing peptide in the heparin neutralizer may be in a range of 5 to 200 μg/mL.

The present disclosure further provides use of the heparin neutralizing peptide or the heparin neutralizer according to foregoing solutions in the preparation of a medicament for neutralizing heparin.

Preferably, the heparin may include unfractionated heparin and/or low molecular weight heparin (LMWH).

Preferably, the medicament may be in a dosage form of an intravenous injection.

Preferably, based on the mass of the heparin neutralizing peptide in the medicament, an administration dose of the medicament may be 6-8 times the mass of heparin to be neutralized.

The present disclosure provides a heparin neutralizing peptide. The heparin neutralizing peptide includes tandem repeating units. The repeating units each have an amino acid sequence shown in SEQ ID NO: 1, specifically VPGKG, where lysine (K) has a positive charge, the repeating units each have a positive charge, and the heparin neutralizing peptide has a plurality of positive charges. The heparin neutralizing peptide with positive charges can bind to a heparin with negative charges through electrostatic interaction. A binding force between the heparin neutralizing peptide and the heparin is greater than that between the heparin and antithrombin. The heparin neutralizing peptide binds to the heparin to form macromolecular substances, and the macromolecular substances are eliminated from the body by liver metabolism, so that the anticoagulation of the heparin is shielded. Furthermore, the heparin neutralizing peptide provided in the present disclosure can significantly reduce toxic and side effects of neutralizing heparin and reduce hemolysis compared with protamine sulfate in the prior art.

### BRIEF DESCRIPTION OF THE DRAWINGS

FIG. 1 illustrates an anti-factor Xa assay obtained by a heparin neutralizing peptide containing 72 repeating units (hereinafter referred to as K72) in the present disclosure.

FIG. 2 illustrates a hemolytic test obtained by a K72 material in the present disclosure.

### DETAILED DESCRIPTION OF THE EMBODIMENTS

The present disclosure provides a heparin neutralizing peptide, including tandem repeating units, where the repeating units each have an amino acid sequence shown in SEQ ID NO: 1, specifically: VPGKG.

In the present disclosure, lysine (K) in each repeating unit, VPGKG, has a positive charge, the repeating units each have a positive charge, and the heparin neutralizing peptide has a plurality of positive charges. The heparin neutralizing peptide with positive charges can bind to a heparin with negative charges through electrostatic interaction to form macromolecular substances, and the macromolecular substances are eliminated from the body by liver metabolism, so that the anticoagulation of the heparin is shielded.

The present disclosure does not specifically limit the number of the repeating units. In the specific implementation of the present disclosure, the number of the repeating units may preferably be 18-144, and more preferably 36-72.

In the present disclosure, the heparin neutralizing peptide may preferably be obtained by gene recombination technology.

In the present disclosure, a corresponding gene sequence, K18, is designed and synthesized according to a VPGKG peptide repeating unit sequence, and restriction endonuclease sites (for example: NdeI, XhoI, Van911, and Bg/I include the restriction endonuclease sites but are not limited to these four restriction endonuclease sites) are designed at either end; the K18 gene sequence is inserted into an Escherichia coli expression vector PET-25b or other vectors and transformed into engineering E. coli BL21 for the expression of K18 peptide. To obtain gene sequences of K36, K72, and K144, based on the K18 sequence, the K18 is amplified into the K36 using properties of the Van911 and Bg/I restriction endonucleases, and the K72 and the K144 are further obtained. Likewise, the gene sequences of the K36, the K72 and the K144 are inserted into the E. coli expression vector PET-25b vector and transformed into the engineering E. coli BL21 for the expression of K36, K72 and K144 peptides.

The present disclosure provides a heparin neutralizer, including a buffer and the heparin neutralizing peptide according to the foregoing solution.

In the present disclosure, the buffer may preferably include a PBS. In the present disclosure, the PBS uses water as a solvent, and preferably includes raw materials at the following concentrations: 2.5 mM NaH₂PO₄, 7.5 mM Na₂HPO₄, and 150 mM NaCl; the PBS may preferably be at pH 7.4.

In the present disclosure, a concentration of the heparin neutralizing peptide in the heparin neutralizer may preferably be in a range of 5 to 200 μg/mL.

In the present disclosure, the heparin neutralizer is prepared by: mixing the buffer and the heparin neutralizing peptide to obtain the heparin neutralizer.

The present disclosure further provides use of the heparin neutralizing peptide or the heparin neutralizer according to foregoing solutions in the preparation of a medicament for neutralizing heparin.

In the present disclosure, the heparin may preferably include unfractionated heparin and/or LMWH. In the present disclosure, a molecular weight of the unfractionated heparin may be greater than 5,000 Da, and that of the LMWH may be 4,000-6,000 Da.

In the present disclosure, the medicament may preferably be in a dosage form of an intravenous injection.

In the present disclosure, based on the mass of the heparin neutralizing peptide in the medicament, an administration dose of the medicament may preferably be 6-8 times the mass of heparin to be neutralized.

The technical solutions of the present disclosure will be clearly and completely described below with reference to the examples of the present disclosure. Apparently, the described examples are only a part of, not all of, the examples of the present disclosure. Based on the examples of the present disclosure, all other examples obtained by those of ordinary skill in the art without creative efforts shall fall within the protection scope of the present disclosure.

### Example 1

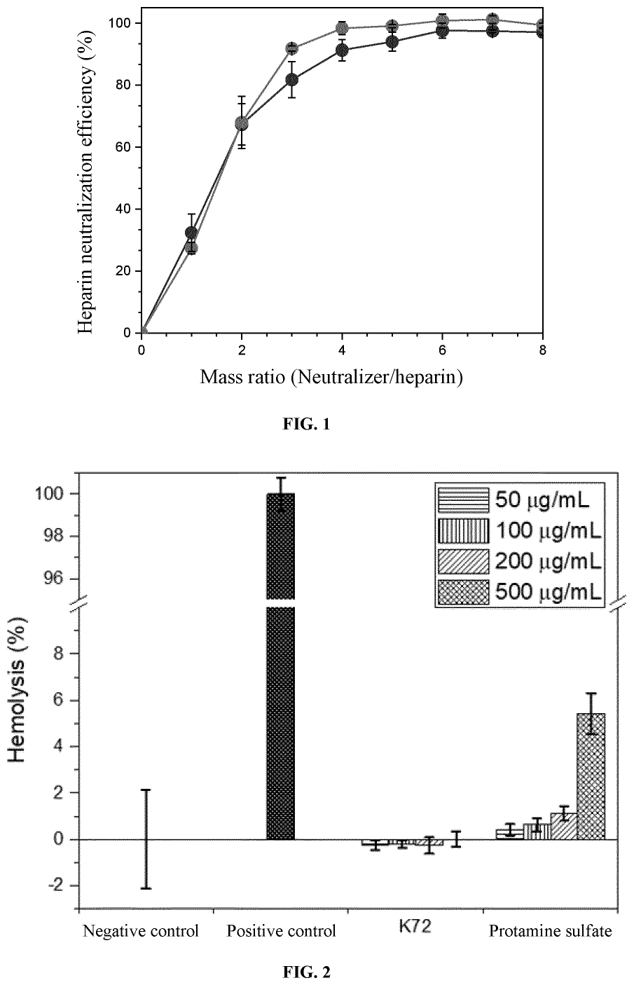

Separately, 0, 10, 20, 30, 40, 50, 60, 70, and 80 μL of 2 μg/mL K72 (the number of repeating units VPGKG was 72) in PBS were mixed with 10 μL of 2 μg/mL unfractionated heparin in PBS into centrifuge tubes. Subsequently, an appropriate quantity of PBS was added to each centrifuge tube, so that 100 μL of a solution was finally contained in each centrifuge tube. All centrifuge tubes were shaken and blended for 1 min, and 40 μL of each solution was pipetted to determine the concentration of unneutralized heparin by anti-factor Xa assay. Table 1 and FIG. 1 show the results of the anti-factor Xa assay obtained in this example. The assay results showed that 97% of the heparin was neutralized when the mass ratio of K72 to heparin was 6.

TABLE 1
Results of anti-factor Xa assay

| | Heparin neutralization | |
|---|---|---|
| Mass ratio | efficiency (%) | |
| (Neutralizer/ | | Protamine |
| heparin) | K72 | sulfate |
| 0 | 0 | 0 |
| 1 | 35.35 ± 6.01 | 27.36 ± 1.86 |
| 2 | 67.34 ± 6.71 | 67.98 ± 8.37 |
| 3 | 81.76 ± 5.79 | 91.82 ± 0.85 |
| 4 | 91.30 ± 3.47 | 98.37 ± 2.08 |
| 5 | 94.01 ± 3.07 | 99.10 ± 0.48 |
| 6 | 97.63 ± 2.39 | 100.78 ± 2.26 |
| 7 | 97.47 ± 0.47 | 101.18 ± 1.28 |
| 8 | 97.17 ± 1.04 | 99.34 ± 0.70 |

Fresh human blood samples were used in the hemolytic test. Separately, 1 mL of blood was placed in a centrifuge tube and centrifuged, and the serum supernatant was carefully removed. Red blood cells were then washed with the same volume of PBS as the serum and centrifuged, and the supernatant was removed. This washing operation was repeated three times until the supernatant became clear. The washed red blood cells were diluted to 50 mL with PBS. Subsequently, 180 μL of the diluted red blood cell solution was pipetted into each well of a 96-well plate. Separately, 20 μL of 0.5, 1, 2, or 5 mg/mL K72 or protamine sulfate in PBS was added to the 96-well plate containing red blood cells, and finally the concentration of K72 or protamine sulfate was obtained as 0.05, 0.1, 0.2, and 0.5 mg/mL. 20 μL of PBS was used as a negative control, and 20 μL of 10% Triton X-100 was used as a positive control. The resulting 96-well plate was left to stand in a 37° C. incubator for 1 h, and residual red blood cells were pelleted by centrifugation. 100 μL of the supernatant was pipetted from the centrifuged 96-well plate and correspondingly placed in a new 96-well plate, and the absorbance (A) of each well at a wavelength of 540 nm was read on a multifunctional microplate reader. The hemolysis ratio was calculated according to the following formula:

$$\% Hemolysis ratio =$$

$$[ ( {A}_{Sample} - {A}_{Negative control} ) / ( {A}_{Positive control} - {A}_{Negative control} ) ] \times 100$$

FIG. 2 illustrates the results of the hemolytic test obtained in this example. The test results showed that hemolysis had not been induced yet when the K72 concentration reached 0.5 mg/mL, while protamine sulfate caused 5% hemolysis at the same concentration.

The foregoing description is merely preferred implementation of the present disclosure. It should be noted that several improvements and modifications can be made by a person of ordinary skill in the art without departing from the principles of the present disclosure, and these improvements and modifications should be construed as falling within the protection scope of the present disclosure.

1. A heparin neutralizing peptide, comprising tandem repeating units, wherein the repeating units each have an amino acid sequence shown in SEQ ID NO: 1.

2. The heparin neutralizing peptide according to claim 1, wherein a number of the repeating units is 18, 36, 72, or 144.

3. A heparin neutralizer, comprising a buffer and the heparin neutralizing peptide according to claim 1.

4. The heparin neutralizer according to claim 3, wherein the buffer comprises a phosphate-buffered saline (PBS).

5. The heparin neutralizer according to claim 3, wherein a concentration of the heparin neutralizing peptide in the heparin neutralizer is in a range of 5 to 200 μg/mL.

6. Use of the heparin neutralizing peptide according to claim 1 in the preparation of a medicament for neutralizing heparin.

7. The use according to claim 6, wherein the heparin comprises unfractionated heparin and/or low molecular weight heparin (LMWH).

8. The use according to claim 6, wherein the medicament is in a dosage form of an intravenous injection.

9. The use according to claim 7, wherein based on the mass of the heparin neutralizing peptide in the medicament, an administration dose of the medicament is 6-8 times the mass of heparin to be neutralized.
